(19) United States
(12) Patent Application Publication
(10) Pub. No.: US 2021/0322601 A1
(43) Pub. Date: Oct. 21, 2021

(54) STERILIZING MATERIAL OR DEVICE AND METHOD OF USING THE SAME

(71) Applicant: Eric F. BERNSTEIN, Gladwyne, PA (US)

(72) Inventor: Eric F. BERNSTEIN, Gladwyne, PA (US)

(21) Appl. No.: 17/236,389

(22) Filed: Apr. 21, 2021

Related U.S. Application Data
(60) Provisional application No. 63/013,516, filed on Apr. 21, 2020.

(51) Int. Cl. A61L 2/10; C09K 11/77; A61L 2/08; A61L 2/26; C09D 5/14; C09D 5/08; C09D 7/61

(52) U.S. Cl. CPC A61L 2/10; C09K 11/7777; A61L 2/088; A61L 2202/11; C09D 5/14; C09D 5/08; C09D 7/61; A61L 2/26

(57) ABSTRACT

Materials and devices comprising at least one UV emitting device or material for emitting energy having a wavelength ranging from 100-400 nm, and at least one material that exhibits sterilizing properties, the material comprising a sterilizing metal compound, oxide, polymer or combinations thereof. A method of sterilizing a surface using the disclosed materials and devices are also disclosed.

### PRIORITY

This application claims the benefit of priority to U.S. Provisional Application No. 63/013,516, filed Apr. 21, 2020, which is incorporated herein by reference in its entirety.

### FIELD OF USE

The present disclosure generally relates to a sterilizing materials and devices for killing microorganisms on surfaces, such as touchscreens that directs UV energy, such as UVB and/or UVC to the surface. The present disclosure also relates to sterilizing covers that contain devices for generating germicidal UV energy, such as comprising organic and/or inorganic phosphors that convert an incident radiation to a different radiation, or combinations thereof. The present disclosure also relates to methods of sterilizing the surface of a touchscreen by elimination, inactivation or reduction of pathogens including viruses, bacteria, fungi, yeast or prions.

### BACKGROUND

Touchscreen technology was first described in the 1960's. By 1975 the U.S. Patent Office issued one of the first patents, U.S. Pat. No. 3,911,215 on a resistive touchscreen to Hurst et al. Since that time, touchscreens have become common in a variety of electronic devices such as smart phones, tablets, personal computers, point-of-sale (POS) systems, kiosks, digital appliances, and other functional electronics, including automated teller machines (ATMs).

The ubiquitous use of touchscreens necessarily requires increased human-computer interaction particularly public-use touchscreens, such as self-check in kiosks at airports and train stations to self-check out stations at grocery stores. Even if public touchscreens are wiped down frequently throughout the day cleaning, harmful bacteria can remain on touchscreen surfaces for days.

As touchscreen technology becomes a more integral part of our daily lives, our exposure to germs increases as these surfaces are touched by countless people every day, each time collecting germs and bacteria from users, such as Staphylococcus (staph), and Enterococcus faecalis (E. faecalis) which is notorious for causing hospital-acquired infections (HAIs). A majority of the germs found on touchscreen surfaces in public areas is likely the result of poor hand hygiene. Studies have shown that about 34% of Americans do not wash their hands after using the restroom. This might explain the results of research done by Insurance Quotes which indicates the average airport self-check-in screen at contained 253,857 colony-forming unit (CFU). In comparison, on average only 172 CFU are found on public toilet seats.

In view of the foregoing, there is a need to be able to continuously and automatically sterilize the surface of touchscreens. It is known that UV-C light is “germicidal” because it can deactivate the DNA of bacteria, viruses and other pathogens and thus destroys their ability to multiply and cause disease without the need for heat or chemicals. The short wavelength associated with UVC energy, specifically between about 250 to about 260 nm, and lower, provides the highest germicidal effectiveness, and thus is lethal to a variety or microorganisms, including the most common molds, virus, and bacteria, such as Salmonella, Staphylococcus, Streptococcus, Legionella, Bacillus, dysentery, infectious hepatitis, influenza, coronavirus and rotavirus. Accordingly, there is a need for a more economical and safer method of using the powerful germicidal effects of UV technology.

To solve at least one of the foregoing problems, there is described a touchscreen cover that contains at least one mechanism for generating UV energy, such as UVB and/or UVC energy on the surface of the touchscreen. To that end, there is described a cover for touchscreens, such as a plastic or glass cover, that directs UVC on the user facing surface of a touchscreen. In one embodiment, UVC energy is generated from the up-conversion of inherent blue light emitted from the touchscreen device itself by organic or inorganic phosphors contained in the cover.

### SUMMARY OF INVENTION

Thus, the present disclosure is directed to a material or device for converting electromagnetic energy to ultraviolet radiation or electromagnetic radiation of shorter wavelengths for the purpose of sterilization by elimination, inactivation or reduction of pathogens including viruses, bacteria, fungi, yeast or prions.

In one embodiment, there is disclosed a sterilizing material or device, comprising: at least one UV emitting device or material for emitting energy having a wavelength ranging from 100-400 nm, and at least one material that exhibits sterilizing properties, the material comprising a sterilizing metal compound, oxide, polymer. In one embodiment, the the at least one UV emitting device is selected from a UV emitting diode or a UV emitting material, wherein the UV emitting material comprises a composition comprising at least one phosphor capable of converting an initial electromagnetic energy (A) to a different electromagnetic energy (B), said different electromagnetic energy (B) comprising UVB, UVC, or combinations thereof.

In another embodiment, there is disclosed a method of sterilizing a surface by exposing it to UV radiation ranging from 100-400 nm, the method comprising: exposing a material to UV radiation generated from at least one UV emitting device or material for emitting energy having a wavelength ranging from 100-400 nm, wherein exposing is performed for a time sufficient to deactivate or kill at least one microorganism chosen from bacteria, virus, mold, protozoa, and yeast. Non-limiting examples of the bacteria that can be killed according to the disclosed method include E. coli, Salmonella, Staphylococcus, Streptococcus, Legionella, Bacillus, Rhodospirillum, Mycobacterium, Clostridium, dysentery, and tuberculosis. Non-limiting examples of the virus that can be killed according to the disclosed method includes bacteriophage, coxsackie, infectious hepatitis, influenza, coronavirus, and rotavirus.

### DETAILED DESCRIPTION OF THE INVENTION

### Definitions

Certain terms used herein are defined below:

“Up-converting” refers to the ability to convert electromagnetic energy to a higher energy or shorter wavelength.

“Down-converting” refers to the ability to convert electromagnetic energy to a lower energy or longer wavelength.

“At least one” as used herein means one or more and thus includes individual components as well as mixtures/combinations.

A “film,” as used herein, refers to a continuous coating, i.e., a coating without holes visible to the naked eye, which covers at least a portion of the substrate to which the composition was applied. Further, a film, as used herein, may have any thickness and is not restricted to a thin coating.

“Film-forming polymer” as used herein means a polymer which, by itself or in the presence of a film-forming auxiliary, is capable, after dissolution in at least one solvent, of forming a film on the substrate to which it is applied once the at least one solvent evaporates.

“Polymers” as defined herein comprise copolymers (including terpolymers) and homopolymers, including but not limited to, for example, block polymers, cross linked polymers, and graft polymers.

“UV radiation” as defined herein encompasses radiation having a wavelength ranging from 100-400 nm, specifically including UVC (200-290 nm), UVB (290-320 nm) and UVA (320-400 nm) radiations.

“UV emitter” as defined herein includes a material or device for generating UV radiation. Non-limiting examples of materials for emitting UV radiation include at least one phosphor described herein capable of converting an initial electromagnetic energy (A) to a UV energy. Non-limiting examples of a device for emitting UV radiation include UV generating diodes, such as a or liquid crystal diode (LCD) or a light emitting diode (LED).

“Optical communication” means light is able to be transmitted from a light emitting source to another location, such as the surface of a cover. Optical communication does not require physical, electrical or thermal contact.

“Contact sterilization,” means the killing of microorganisms when they come into contact with a surface, such as a touchscreen, that is being treated with UV radiation as described herein.

“Release sterilization,” means the killing of microorganisms by releasing material onto a contaminated surface containing microorganisms, such as by applying a spray, paint or the like to the surface.

It is to be understood that both the foregoing general description and the following detailed description are exemplary and explanatory only and are not restrictive of the invention, as claimed. Reference will now be made in detail to exemplary embodiments of the present invention.

Ultraviolet (UV) radiation is known as a highly effective means of destroying microorganisms. For example, typical UV radiation that can be used in the described devices include UVC (200-290 nm), UVB (290-320 nm) and UVA 320-400 nm. In one embodiment, the desired wavelength would range from 240 nm to 260 nm, such as 254 nm. It is known that this range is highly effective in killing bacteria, molds, protozoa, yeasts, and viruses on surfaces, such as touchscreens. Methods of killing the pathogen from exposure to UVC include fatal harm to microbial DNA by triggering adjacent thymine molecules to dimerize, thereby disrupting DNA and RNA replication.

As described herein, there are multiple ways to sterilize the surface of a touchscreen with UV energy. In one embodiment, there is described a cover for a touchscreen comprising a UV emitter. In one embodiment, the UV emitter is a material, such as a phosphor that converts an initial incident radiation to UV radiation.

A phosphor is a substance that exhibits the phenomenon of phosphorescence, or a sustained glowing after exposure to light or energized particles such as electrons. Phosphors have a finite emission time, with persistence being inversely proportional to wavelength. Because the persistence of the phosphor increases as the wavelength decreases, it is known that red and orange phosphors do not have sufficiently long glow times.

The organic and inorganic phosphors used in the present invention differ from these traditional phosphors in that they have an indefinite glow time. In addition, they have the ability to transfer electromagnetic energy of one frequency to a higher frequency (referred to as “up-converting”) or to a lower frequency (referred to as “down-converting”), depending on the rare earth metal used. A description of such phosphors is provided U.S. Pat. No. 5,698,397, which is herein incorporated by reference. This patent describes the use of such phosphors for biological and other assays.

Up-converting crystals, which take light or electromagnetic radiation of one frequency and convert it to light of a higher frequency (thus shorter wavelength), appear to contradict a basic law of physics directed to conservation of energy. However, two, four or more photons of a lower frequency or longer wavelength are converted into a single photon of higher frequency or shorter wavelength. Thus, a number of photons of lower energy combine to produce one photon of higher energy. These compounds can emit visible light when irradiated with infra-red light.

In contrast, down-converting crystals take light or electromagnetic radiation of one frequency and convert it to light of a lower frequency (thus longer wavelength). These compounds can emit red or IR light when irradiated with UV or visible light.

Phosphors are usually made from a suitable host material, to which an activator is added. Suitable activators that may be used in the present invention include ytterbium, erbium, thulium, holmium, and combinations of these materials. Non-limiting examples of activator couples include ytterbium/erbium, ytterbium/thulium, and ytterbium/holmium.

Generally, host materials comprise oxides, halides, sulfides, and selenides of various rare earth metals. Suitable phosphor host materials that may be used in one embodiment of the present invention include gadolinium, yttrium, lanthanum, and combinations of these materials. Particular non-limiting embodiments of such crystal matrices which may comprise the host material include oxy-sulfides, oxy-fluorides, oxychlorides, or vanadates of various rare earth metals.

Non-limiting embodiments of the organic and/or inorganic phosphors that can be used as host materials in the present disclosure include sodium yttrium fluoride (NaYF₄), lanthanum fluoride (LaF₃), lanthanum oxysulfide (La₂O₂S), yttrium oxysulfide (Y₂O₂S), yttrium fluoride (YF₃), yttrium gallate, yttrium aluminum garnet (YAG), gadolinium fluoride (GdF₃), barium yttrium fluoride (BaYF₅, BaY₂F₈), gadolinium oxysulfide (Gd₂O₂S), calcium tungstate (CaWO₄), yttrium oxide:terbium (Yt₂O₃Tb), gadolinium oxysulphide: europium (Gd₂O₂S:Eu); lanthanam oxysulphide: europium (La₂O₂S:Eu); and gadolinium oxysulphide: promethium, cerium, fluorine (Gd₂O₂S:Pr,Ce,F); and generally (YLuScA)PO₄, wherein A is an activator selected from the group of bismuth, praseodymium and neodymium.

Other phosphors which may be used in the present composition, along with their characteristic absorption colors (and wavelengths) include, are not limited to: Gd₂O₂S:Tb (P43), green (peak at 545 nm); Gd₂O₂S:Eu, red (627 nm); Gd₂O₂S:Pr, green (513 nm); Gd₂O₂S:Pr,Ce,F, green (513 nm); Y₂O₂S:Tb (P45), white (545 nm); Y₂O₂S:Tb red (627 nm); Y₂O₂S:Tb, white (513 nm); Zn(0.5)Cd(0.4)S:Ag green (560 nm); Zn(0.4)Cd(0.6)S:Ag (HSr), red (630 nm); CdWO₄, blue (475 nm); CaWO₄, blue (410 nm); MgWO₄, white (500 nm); Y₂SiO₅:Ce (P47), blue (400 nm); YAlO₃:Ce (YAP), blue (370 nm); Y₃Al₅O₁₂:Ce (YAG), green (550 nm); Y₃(AI,Ga)₅O₁₂:Ce (YGG), green (530 nm); CdS:In, green (525 nm); ZnO:Ga, blue (390 nm); ZnO:Zn (P15), blue (495 nm); (Zn,Cd)S:Cu,AI (P22G), green (565 nm); ZnS:Cu,AI,Au (P22G), green (540 nm); ZnCdS:Ag,Cu (P20), green (530 nm); ZnS:Ag (P11), blue (455 nm); Zn₂SiO₄:Mn (P1), green (530 nm); ZnS:Cu (GS), green (520 nm); and the following crystals that emit in a UV-C range, e.g., from 200 to 280 nm, such as from 225 to 275 nm: YPO₄:Nd; LaPO₄:Pr; (Ca,Mg)SO₄:Pb; YBO₃:Pr; Y₂SiO₅:Pr; Y₂Si₂O₇:Pr; SrLi₂SiO₄:Pr,Na; and CaLi₂SiO₄: Pr.

In one embodiment, the organic and/or inorganic phosphors are present in the disclosed composition in an amount effective to convert electromagnetic radiation of a frequency (A) to a higher frequency (B). While in theory, the up-converting crystals of this embodiment can convert any electromagnetic energy to a higher energy (or shorter wavelength), in one embodiment, the electromagnetic radiation of frequency (A) comprises infrared or visible light, and the frequency (B) comprises ultraviolet (UV) radiation chosen from UVA, UVB, and UVC.

The organic and/or inorganic phosphors may be present in the disclosed composition in an amount ranging from 0.01% to 60% by weight, relative to the total weight of the composition, such as from 0.1% to 30% or even 1% to 15% by weight, relative to the total weight of the composition.

In one embodiment, the disclosed composition may further comprise an activator for the organic and/or inorganic phosphors, such as a ytterbium containing activator. Non-limiting examples of the ytterbium containing activator include ytterbium/erbium, ytterbium/thulium, ytterbium/terbium, and ytterbium/holmium.

The organic and/or inorganic phosphors according to the present disclosure typically have an average particle size ranging from 1 nm to 1 cm, such as from 1 nm to 1 mm, from 2 nm to 1000 nm, from 5-100 nm, or even 10-50 nm. The concentration of the organic and/or inorganic phosphors in the inventive composition as well as in the above-defined regions and the size of the organic and/or inorganic phosphors can be measured by methods known for such which are well known in the art. For example, x-ray diffraction (XRD), scanning electron microscopy (SEM), transmission electron microscopy (TEM), and/or BET surface area analysis may be used.

The organic and/or inorganic phosphors according to the present disclosure are typically synthesized from rare-earth doped phosphorescent oxide particles having the previously described sizes. The method further provides for homogeneous ion distribution through high temperature atomic diffusion.

A solid-phase precursor composition (hereinafter referred to as “the precursor composition”) is prepared by mixing one or more rare earth element dopant precursor powders with one or more oxide-forming host metal powders. Stoichiometric amounts of host metal and rare earth element are employed to provide rare earth element doping concentrations in the final particle of at least 0.5 mol % up to the quenching limit concentration.

In one embodiment, the quenching limit concentration is about 15-18 mol % for europium-doped Y₂O₃ nanoparticles, while it is about 10 mol % for erbium-doped Y₂O₃ nanoparticles. Also, for Yb and Er-codoped Y₂O₃ nanoparticles, the quenching limit depends upon the ratio of Yb:Er.

The rare earth element dopant precursor powders include, but are not limited to organometallic rare earth complexes having the structure:

RE(X)₃

wherein X is a trifunctional ligand and RE is a rare earth element. Any rare earth element or combinations thereof can be used (i.e., europium, cerium, terbium, dysprosium, holmium, erbium, thulium, ytterbium, lutetium) with particular mention being made to europium, cerium, terbium, holmium, erbium, thulium and ytterbium, as well as the following combinations: ytterbium and erbium, ytterbium and holmium and ytterbium and thulium.

Strontium can also be used, and for purposes of the present invention, rare earth elements are defined as including strontium. are earth element dopant precursor powders include Yb(TMHD)₃, Er(TMHD)₃, Ho(TMHD)₃, Tm(TMHD)₃, erbium isopropoxide (C₉H₂₁O₃Er), ytterbium isopropoxide (C₉H₂₁O₃Yb), and holmium isopropoxide (C₉H₂₁O₃Ho).

Examples of trifunctional ligands include tetramethylheptanedionate (TMHD), isopropoxide (IP), and the like.

The oxide forming host metal can be, but is not limited to, lanthanum, yttrium, lead, zinc, cadmium, and any of the Group II metals such as, beryllium, magnesium, calcium, strontium, barium, aluminum, radium and any mixtures thereof or a metalloid selected from silicon, germanium and II-IV semi-conductor compounds. Oxide-forming host metal powders include Y(TMHD)₃, AI(TMHD)₃, Zr(TMHD)₃, Y(IP), and Ti(IP).

The rare earth element dopant precursor powder and oxide-forming host metal powders are mixed to form the precursor composition, and vaporized. An inert carrier gas, such as, but not limited to, nitrogen, argon, helium, and mixtures thereof, transports the vaporized precursor composition to a low pressure combustion chamber that houses a flame.

The flame produces active atomic oxygen via chain-initiation reaction of

H+O₂=OH+O (i)

A high concentration of oxygen in the flame activates and accelerates the oxidation of rare-earth ions and host materials through a series of reactions:

R+O→RO; (ii)

RO+O→ORO; and (iii)

ORO+RO→R₂O₃ (iv)

Reactions (ii) through (iv) are much faster than the oxidation reaction in low temperature processing represented by the reaction below;

2R+3/2O₂=R₂O₃ (v)

The reaction represented by formula (v) has a much higher energy barrier than the reactions in formulae (i)-(iv) in which radicals formed in flames diffuse and help produce faster ion incorporation.

Generally, in flame spray pyrolysis a higher flame temperature increases particle sintering and agglomeration. However, in one embodiment of the present invention, spherical, discrete particles are formed. It is proposed that in addition to residence time, the initial size of the vapor-phase particles in the vaporized precursor composition and the precursor itself are the dominant factors that determine final particle size. As the vaporized precursor composition passes through the flame, it directly reacts and releases heat to the flame increasing flame temperature. Thus, a shorter flame residence time is needed, which allows for the production of smaller particles.

Temperatures ranging from about 1800 to about 2900° C. are used in one embodiment, with temperatures ranging from about 2200 to about 2400° C. being particularly noted. Temperatures within this range produce monodispersed rare earth doped activated oxide nanoparticles without significant agglomeration having an essentially uniform distribution of rare earth ions within the particles. Actual residence time will depend upon reactor configuration and volume, as well as the volume per unit time of vaporized precursor composition delivered at a given flame temperature. Cubic phase particles are obtained having an average particle size ranging from 5 to 50 nanometers, such as from 10 to 20 nanometers. Until recently, it was not possible to obtain activated cubic phase particles on a nanoscale. The particles also exhibit quenching limit concentrations heretofore unobtained.

The flame temperature can be manipulated by adjusting the flow rates of the gas(es). For example, the temperature of the flame can be increased by increasing the methane flow rate in a methane/oxygen gas mixture. Guided by the present specification, one of ordinary skill in the art will understand without undue experimentation how to adjust the respective flow rates of reactive gas(es) and inert carrier gas to achieve the flame temperature producing the residence time required to obtain an activated particle with a predetermined particle size.

Any reactive gas can be used singularly or in combination to generate the flame for reacting with the vaporized precursor composition, such as, but not limited to, hydrogen, methane, ethane, propane, ethylene, acetylene, propylene, butylenes, nbutane, iso-butane, n-butene, iso-butene, n-pentane, iso-pentane, propene, carbon monoxide, other hydrocarbon fuels, hydrogen sulfide, sulfur dioxide, ammonia, and the like, and mixtures thereof.

A hydrogen flame can produce high purity nano-phosphors without hydrocarbon and other material contamination. In the depicted embodiments, the flame length determines particle residence time within the flame. Higher temperatures produce satisfactory nanoparticles with shorter flames. Flame length is similarly manipulated by varying gas flow rates, which is also well understood by the ordinarily skilled artisan. Increasing the flame length increases the residence time of the particles in the flame allowing more time for the particles to grow. The particle residence time can be controlled by varying the different flow rates of the gases and is readily understood by one of ordinary skill in the art guided by the present specification.

The compositions according to the invention further comprises at least one organic or inorganic media in or on which the disclosed phosphors are dispersed. In one embodiment, the organic media comprises a plastic resin, such as thermoplastic elastomers, high temperature plastics, and engineering thermoplastics. Non-limiting examples of such resins include a polymer or co-polymer of polyvinyl chloride (PVC), acrylonitrile butadiene styrene (ABS), olefin, polycarbonate, styrene, nylon, and acetal.

In embodiments described herein, there is described compositions that further include materials that enhance sterilization, such as silver and copper compounds and ions, or compounds that generate free radicals, such as zinc oxide or titanium oxides, namely TiO₂, or combinations thereof.

Regarding TiO₂, this compound can be used in combination with a UV emitter described herein, to reduce surface bacterial and viral particles, including particles in contact with the touch screen described herein. One benefit of using a TiO₂ coating in combination with one or more UV emitters described herein is the coating is transparent and does not affect the use of a touchscreen.

It is possible to obtain an intimate mixture of the described phosphors and resins by mixture them in a dry state and subsequently compounding them, which may be followed by forming them into desired shapes using known plastic forming techniques, such as injection molding.

The resins described herein are well-known to be formed into a variety of plastics, including clear covers and shields. When compounded in or coated on such a plastic material, it would be possible to achieve localized UVC treatment. The plastic composition used in such applications include polyvinyl chloride, polyolefin or polyester.

In one embodiment, the polymers used herein may in themselves already have antimicrobial properties, such as polyammonium salts or polyethylene glycol, which may be used alone or in combination with the UV emitters des cribbed herein. Such polymers may be used to produce soft coatings for metals, glass and ceramics that are adjacent to and in proximate vicinity to a touchscreen. These materials are UV-resistant and can even provide protection against corrosion.

Other forms of antimicrobial materials or self-sterilizing polymers include elastomeric polymers with highly effective, broad-spectrum antimicrobial properties. In these embodiment, the polymer's antimicrobial properties stem from its unique molecular architecture, which attracts water to a sequence of repeat units that are chemically modified (or functionalized) with sulfonic acid groups.

In one embodiment, compounding includes providing a material matrix, such as plastic materials, with antimicrobial properties while they are still being manufactured when additives are included in the process. For example, in one embodiment, germicidal substances are incorporated in polyethylene, polypropylene or polyamide, where they then have a disinfectant function thanks to the ongoing release of ions, such as with zinc oxide, titanium oxides (TiO₂), copper compounds or silver compounds.

With regarding to the use of silver compounds, it is proposed to use silver ions that diffuse from the material matrix to the surface and have an antimicrobial effect.

There is also disclosed an cover, such as a glass or plastic piece, for covering a touchscreen. For example, in one embodiment, there is disclosed a plastic piece for covering a touchscreen. In this embodiment, the device comprising the touchscreen is capable of emitting the longer wavelength radiation from the inherent backlighting of the device. This longer wavelength radiation will activate the crystals to create UV-C which impinges the surface of the touchscreen causing it to be disinfected. In one embodiment, the cover further comprises a coating on at least one of the interior or exterior walls that blocks the UV-C from exiting the plastic cover, thus preventing UVC from reaching a user of the touchscreen. In one embodiment, the coating for containing the UVC comprises a thin metal layer, or an oxide that is transparent or translucent to the initial energy A but that prevent UVC from exiting the cover, such as MgO, TiO₂, SiO₂ and Al₂O₃. As further described below, these materials, namely TiO₂, have the additional benefit of providing sterilization and purification benefits when excited by ultraviolet light. These unique properties allow the described material to be used as a surface coating that will further kill microorganisms such as bacteria and viruses.

In another embodiment, the UV emitter is a device that generates UV radiation such as diode, including a light emitting diode (LED) or a liquid crystal diode (LCD). These types of diodes have several advantages that make them particularly useful for covers in touchscreen devices, including improved durability, and flexibility that allows them to fit into touchscreens, efficiency of power usage.

In one embodiment, there is described a cover for sterilizing a touchscreen, the cover comprising at least one LED module that is coupled to a cover for a touchscreen, such that a UV-emitting surface is in optical communication with a surface of the touchscreen cover that faces the user. In an embodiment, the LED module is included or embedded in the borders, edges, and/or boundaries of the touchscreen cover by at least one sealing layer.

In an embodiment, the sealing layer may comprise a UV reflective material, such that any light back-reflected from the light-emitting surface of the LED module may be at least partially conserved by re-reflection in the direction of the light emitting surface. In one non-limiting embodiment, the sealing layer may comprise a UV resistant organic material, silicone or silicone composites, a fluoropolymer or its composites. The sealing layer may for example be applied by dispensing or transfer molding, although it will be clear to the skilled person that other application techniques may also be used.

Translucent window element may, by way of non-limiting example, be composed of Polycrystalline Alumina (PC A) materials, such as for example Spinel (MgAl204), AION, or sapphire. However, other suitable translucent ceramic materials may also be used.

In one embodiment, the at least one LED module may be coupled with the translucent widow element such the LED is embedded within the body of the window element. In this embodiment, the light-emitting top surface of the LED is in direct physical contact with surfaces of the translucent window element. As a result, light exiting the top surface of the LED may be transmitted directly to the corresponding engaging surface of the window element, without propagating through any intermediary or interstitial layers.

In one embodiment, the LED module is coupled to the translucent window and not embedded therein. For example, the LED module and the translucent window are solidly adhered with an adhesive layer.

It is envisioned that touchscreens covered with the disclosed cover can undergo almost continuous germicidal treatment just by nature of it being exposed to visible light, whether ambient or for artificial.

There is also disclosed a method of sterilizing a touchscreen by exposing it to UVC radiation or radiation of a shorter wavelength, the method comprising: exposing to long-wave ultraviolet, visible or infrared light, a composition comprising, in an organic or inorganic media, at least one phosphor capable of converting said visible or infrared light to UVC radiation or radiation of a shorter wavelength, such as x-ray or gamma rays. In this embodiment, exposing is performed for a time sufficient to deactivate or kill at least one microorganism, including those chosen from bacteria, virus, mold, protozoa, and yeast.

While the foregoing embodiments focus on uses related to touchscreens and their related devices, other uses and applications can also benefit from the combinations described herein. The resins described herein are well-known to be formed into a variety of plastic consumer and household goods, including hair combs, toothbrushes (and bristles), toilet seats, as well as in all types of food packaging.

Food packaging made from the inventive composition makes it possible to reduce the occurrence of common food bacteria, such E. coli and Salmonella, simply by exposing it to light, even while sitting on a store shelf. Thus, it may be possible to extend the life of a product by using packaging made from the disclosed composition.

In another embodiment, the media is inorganic and comprises a ceramic, metal or fabric. For example, the inorganic media may be ceramic and comprise glass or porcelain when the end-use is for a kitchen or bathroom fixture. Use of such compositions make it possible to form eating and cooking utensils that are resistant to common food bacteria, such E. coli and Salmonella.

In one embodiment, the disclosed composition is used in a medical device. Non-limiting examples of such medical devices include intralumen devices, such as stents, guidewires, and embolic filters. Common materials that can be used in such intralumen devices include metals chosen from stainless steel or an alloy of nickel and titanium, commonly referred to as “NiTinols.”

When coated on such devices, it would be possible to achieve localized UVC treatment even when such a device is inserted in the body. For example, by exposing the body to infrared (IR) radiation, which passes through the body, the IR radiation will convert to UVC radiation or radiation of a shorter wavelength such as x-rays or gamma rays when it comes into contact with coated devices. This localized treatment will inhibit abnormal cell growth within the body, commonly referred to as “restenosis”, which remains a major limitation of percutaneous coronary intervention (PCI).

Other non-limiting examples of medical devices that may benefit from being coated with the inventive composition include needles, catheters, such as intravenous catheters and urinary catheters, surgical instruments, material to be implanted into the body to repair or replace blood vessels, or to be implanted as mesh as in hernia repair, such as polytetrafluoroethylene or other fluoropolymer based materials, including those sold under the name Gortex®, valves for the heart or blood vessels, orthopedic devices such as prosthetic joints, ligaments, cartilage and the like, as well as neurosurgical implants, nerve stimulators, deep brain stimulators, and the like.

Another embodiment is directed to containers for collection and/or storage of bodily fluids, such as blood and blood components. The plastic composition used in such containers typically includes a plastic resin that is suitable for contact with blood, such as polyvinyl chloride, polyolefin or polyester. Such containers must be able sterilize the container, which is typically carried out at high temperatures.

It is possible, however, that exposure of such plastic compositions to high temperatures during steam sterilization, may cause degradation of the plastic composition. Degradation presents obvious problems, including a weakening of the overall mechanical strength of the container. To avoid this problem, it is possible to form a flexible sterilizable storage container for blood and blood components.

The plastic composition used in such containers typically includes a plastic resin that is suitable for contact with blood, such as polyvinyl chloride, polyolefin or polyester, that is compounded with the phosphors disclosed herein. In addition to sterilizing the blood storage vessel from the disclosed composition, the blood stored in the vessel and unwanted contaminants located therein, such as hepatitis, can be easily exposed to a lethal dose of radiation. Thus, in one embodiment, there is disclosed a method of treating blood products by exposing blood products that are contained in a vessel made of the composition disclosed herein, to an initial radiation, which when intersected with the disclosed composition, is converted to UVC radiation or electromagnetic radiation of shorter wavelengths, such as x-rays or gamma-rays.

There is also disclosed an cover, such as a glass or plastic piece, for covering an article to be disinfected. For example, in one embodiment, there is disclosed a plastic piece for covering the head of a toothbrush or the diaphragm of a stethoscope. In this embodiment, the plastic cover is capable of emitting the longer wavelength radiation to activate the crystals and creating UV-C within the environment where the article is being disinfected. In one embodiment, the cover further comprises coating on at least one of the interior or exterior walls that blocks the UV-C from exiting the plastic cover, thus preventing UVC from reaching those in the environment outside of the article that is being disinfected. In one embodiment, the coating for containing the UVC comprises a thin metal layer, or an oxide that is transparent or translucent to the initial energy A but that prevent UVC from exiting the cover, such as MgO, TiO2, SiO2 and Al2O3.

In one embodiment, the cover may be used on top of any product described herein, such as a consumer product or medical device chosen from a toothbrush, comb, razor, stethoscope, or dental implant.

In another embodiment, the organic or inorganic media comprises a fabric derived from natural or synthetic fibers or blends of such fibers. Non-limiting examples of the natural fibers comprise cotton, wool, silk, and combinations thereof. Non-limiting examples of the synthetic fibers are chosen from polyesters, polyamides, acrylics, olefins, aramids, such as Kevlar®, polyurethanes, polyethers, such as glycolpolyethylene glycols, Spandex®, vinyl polymers and copolymers, and combinations thereof. The polyesters comprise polyethyleneterephthalate and polypropyleneterephthalate, and the polyamides comprise nylon.

The foregoing natural and synthetic fibers and fabrics made from such fabrics, or combinations of such fabrics, can be impregnated or coated with the inventive compositions, and then formed into a bandage or article for wound treatment. Such a bandage would mitigate the possibility of infection by exposing the wound to germicidal UV treatment.

In another embodiment, the inventive composition may be dispersed on a liquid medium to form a sprayable slurry. This embodiment is particularly suited to retro-fit existing articles to make them germicidal. For example, it is possible to coat an existing article, such as a kitchen counter, by spraying, dipping, or painting onto the article, a composition described herein.

In this embodiment, the organic or inorganic media comprises a liquid for forming a sprayable slurry comprising: (a) at least one polyurethane; (b) at least one acrylic polymer or copolymer; and (c) at least one mineral or organic fillers, the composition optionally containing at least one crosslinking agent. In one embodiment, at least one of (a) to (c) is contained in an aqueous medium.

It is envisioned that consumer products made from or coated with the inventive composition can undergo almost continuous germicidal treatment just by nature of it being exposed to visible light, whether ambient or for artificial. In addition to the foregoing, other advantageous end-uses include, but are not limited to, any article that comes into contact with the general public, including door nobs, handrails, telephones, school desks, seat and arm cushions and headrests on public transportation vehicles.

Other areas that would benefit from the use of articles made from the invention include hospitals and doctor offices. For example, furniture, toys and other articles that have an increased exposure to virus, bacteria, yeast and fungi and can be almost continuously treated by simply exposing them to light.

There is also disclosed a method of sterilizing an article by exposing it to UVC radiation or radiation of a shorter wavelength, the method comprising: exposing to long-wave ultraviolet, visible or infrared light, a composition comprising, in an organic or inorganic media, at least one phosphor capable of converting said visible or infrared light to UVC radiation or radiation of a shorter wavelength, such as x-ray or gamma rays. In this embodiment, exposing is performed for a time sufficient to deactivate or kill at least one microorganism, including those chosen from bacteria, virus, mold, protozoa, and yeast.

In another embodiment, the method may be used to inhibit abnormal cell growth within the body. This is typically used when the composition is in or on an implantable medical device, such as a stent.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and in the attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. The following examples are intended to illustrate the invention without limiting the scope as a result. The percentages are given on a weight basis

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and in the attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. The following examples are intended to illustrate the invention without limiting the scope as a result. The percentages are given on a weight basis.

What is claimed is:

1. A sterilizing material or device, comprising:
at least one UV emitting device or material for emitting energy having a wavelength ranging from 100-400 nm, and
at least one material that exhibits sterilizing properties, said material comprising a sterilizing metal compound, oxide, polymer.

2. The sterilizing material or device of claim 1, wherein the at least one UV emitting device is selected from a UV emitting diode or a UV emitting material, wherein the UV emitting material comprises a composition comprising at least one phosphor capable of converting an initial electromagnetic energy (A) to a different electromagnetic energy (B), said different electromagnetic energy (B) comprising UVB, UVC, or combinations thereof.

3. The sterilizing material or device of claim 2, wherein the UV emitting diode is a light emitting diode (LED), or a liquid crystal diode (LCD).

4. The sterilizing material or device of claim 2, wherein said at least one phosphor is chosen from sodium yttrium fluoride (NaYF₄), lanthanum fluoride (LaF₃), lanthanum oxysulfide (La₂O₂S), yttrium oxysulfide (Y₂O₂S), yttrium fluoride (YF₃), yttrium gallate, yttrium aluminum garnet (YAG), gadolinium fluoride (GdF₃), barium yttrium fluoride (BaYF₅, BaY₂F₈), gadolinium oxysulfide (Gd₂O₂S), calcium tungstate (CaWO₄), yttrium oxide:terbium (Yt₂O₃Tb), gadolinium oxysulphide: europium (Gd₂O₂S:Eu); lanthanum oxysulphide: europium (La₂O₂S:Eu); and gadolinium oxysulphide: promethium, cerium, fluorine (Gd₂O₂S:Pr,Ce,F); YPO₄:Nd; LaPO₄:Pr; (Ca,Mg)SO₄:Pb; YBO₃:Pr; Y₂SiO₅:Pr; Y₂Si₂O₇:Pr; SrLi₂SiO₄:Pr,Na; and CaLi₂SiO₄:Pr.

5. The sterilizing material or device of claim 2, wherein said composition comprises a host lattice represented by the formula (Y_{1-x-y-z}, Luₓ, Sc_{y}, A_{z})PO₄, wherein 0≤x<1 and 0<y≤1 and 0≤z<0.05 and A is an activator.

6. The sterilizing material or device of claim 5, wherein the activator comprises ytterbium, bismuth, praseodymium and neodymium.

7. The sterilizing material or device of claim 6, wherein said activator comprises ytterbium and is chosen from ytterbium/erbium, ytterbium/thulium, ytterbium/terbium, and ytterbium/holmium.

8. The sterilizing material or device of claim 2, wherein the initial electromagnetic radiation of frequency (A) comprises infrared radiation or visible light.

9. The sterilizing material or device of claim 2, wherein said at least one phosphor has an average particle size ranging from 5 nm to 1000 nm.

10. The cover of claim 2, wherein said at least one phosphor is present in an amount ranging from 0.01% to 60% by weight, relative to the total weight of the composition.

11. The sterilizing material or device of claim 2, wherein the composition further comprises a glass or a plastic resin in which the phosphor is embedded or on which it is coated.

12. The sterilizing material or device of claim 11, wherein said plastic resin comprises a polymer or co-polymer of polyvinyl chloride (PVC), polyethylene terephthalate (PET), polymethylmethacrylate (PMMA), polycarbonate (PC), acrylonitrile butadiene styrene (ABS), olefin, styrene, nylon, and acetal.

13. The sterilizing material or device of claim 1, comprising at least one of the following: a sterilizing metal compound chosen from copper and silver, a sterilizing oxide chosen from titanium dioxide and zinc oxide, and a sterilizing polymer chosen from polyammonium salt, polyethylene glycol, and elastomeric polymers modified (or functionalized) with sulfonic acid groups.

14. A method of sterilizing a surface by exposing it to UV radiation ranging from 100-400 nm, said method comprising:
exposing a material to UV radiation generated from at least one UV emitting device or material for emitting energy having a wavelength ranging from 100-400 nm,
wherein said material comprises a sterilizing metal compound, oxide, or polymer, and
said exposing is performed for a time sufficient to deactivate or kill at least one microorganism chosen from bacteria, virus, mold, protozoa, and yeast.

15. The method of claim 14, wherein said bacteria is chosen from: E. coli, Salmonella, Staphylococcus, Streptococcus, Legionella, Bacillus, Rhodospirillum, Mycobacterium, Clostridium, dysentery, and tuberculosis.

16. The method of claim 14, wherein said virus is chosen from bacteriophage, coxsackie, infectious hepatitis, influenza, coronavirus, and rotavirus.

17. The method of claim 14, comprising:
spraying, dipping, or painting onto said article, a composition comprising, in an organic or inorganic media, at least one phosphor capable of converting said UVB, UVA, visible or infrared light to UVC radiation or radiation of shorter wavelength.

18. The method of claim 17, wherein said organic or inorganic media comprises a liquid for forming a sprayable slurry of said composition comprising:
(a) at least one polyurethane;
(b) at least one acrylic polymer or copolymer; and
(c) at least one mineral or organic fillers,
said composition optionally containing at least one crosslinking agent.

19. The method of claim 18, wherein at least one of (a) to (c) is contained in an aqueous medium.
